(19) United States
(12) Patent Application Publication
(10) Pub. No.: US 2023/0194690 A1
(43) Pub. Date: Jun. 22, 2023

(54) A Device For Measuring 3D-Coordinates Associated With An Object

(71) Applicant: Essence Smartcare Ltd., Herzlia Pituach (IL)

(72) Inventors: Barak KATZ, Petach Tikva (IL); Ohad AMIR, Herzlia (IL)

(21) Appl. No.: 17/926,529

(22) PCT Filed: May 21, 2021
(86) PCT No.: PCT/IL2021/050593

§ 371 Date: Nov. 18, 2022

(30) Foreign Application Priority Data
May 21, 2020 (GB) 2008033.9

(51) Int. Cl. G01S 13/46; G01S 7/41

(52) U.S. Cl. CPC G01S 13/46; G01S 7/412; G01S 2013/468

(57) ABSTRACT

A device for measuring 3D coordinates associated with an object in an environment, comprising: a 3D reflected wave measuring device having a 3D coordinate system for measuring 3D coordinates of an object in the environment, the 3D reflected wave measuring device having a first frame of reference; and an orientation sensing device to indicate an orientation of the 3D reflected wave measuring device in an environment for aligning the first frame of reference with a second frame of reference that is associated with the environment. The device may be a fall detector device, although other applications are possible.

### TECHNICAL FIELD

The present invention relates generally to device for measuring 3D coordinates associated with an object in an environment, and a method of using such a device. In particular, embodiments of the invention relate to alignment of the device in an environment.

### BACKGROUND

A 3D reflected wave measuring device can be used to identify the height of locations from which waves are reflected to the measuring device (and detected as reflected wave measurements). This may be achieved by subtracting a vertical coordinate representing a surface (with respect to which the height is to be referenced) from a vertical coordinate that corresponds to the reflection location.

However, for a 3D reflected wave measuring device having a three dimensional coordinate system the reflected wave measuring device may be installed in an orientation that is not aligned with an expected a frame of reference within the environment, which may result in incorrect determinations of heights.

### SUMMARY OF THE INVENTION

The inventors have identified that 3D reflected wave measuring devices have applications in the field of fall detectors or security. As such, determining a correct height metric associated with an object of interest within an environment may be important.

Therefore, the present invention provides a device for measuring 3D coordinates associated with an object in an environment, and the method of using such a device, in accordance with the independent claims appended hereto. Further advantageous embodiments are also defined in the dependent claims appended hereto.

More specifically, the present invention provides a device for measuring 3D coordinates associated with an object in an environment, comprising: a 3D reflected wave measuring device having a 3D coordinate system for measuring 3D coordinates of an object in the environment, the 3D reflected wave measuring device having a first frame of reference; and an orientation sensing device to indicate an orientation of the 3D reflected wave measuring device in an environment for aligning the first frame of reference with a second frame of reference that is associated with the environment.

The orientation sensor advantageously enables the 3D reflected wave measuring device to be aligned correctly to a frame of reference within an environment. This in turn enables a more accurate measurement of 3D reflected wave data points from an object within the environment to be made.

The device may comprise a processor coupled to the orientation sensing device for receiving orientation data indicative of the orientation of the device.

In one embodiment, where a physical correction to the alignment of the device may be made, the device may be connected to one or more actuators for adjusting the orientation of the device in respective one or more axes, and wherein the processor drives the one or more actuators in response to the orientation data to align the first frame of reference to the second frame of reference.

In an alternative embodiment, the processor may output an indication of the orientation of the device in response to the orientation data, the indication for alerting a user at least one of audibly and visually. The indication may indicates at least one of an angular direction and an angle in one or more axes in which the device should be moved in order to align the first frame of reference to the second frame of reference; and/or whether or not the device is aligned.

In a further alternative embodiment, employing a mathematical method of aligning the device, the processor may be configured to align the first frame of reference with the second frame of reference by using the orientation data to compensate for a difference between the first frame of reference and the second frame of reference.

In this embodiment, the processor may configured to compensate for a difference between the first frame of reference and the second frame of reference by applying a rotation matrix to one or more axes of the first frame of reference and/or to one or more coordinates of 3D reflected wave measurements received by the 3D reflected wave measuring device.

The processor may be configured to determine a height metric of an object in the environment relative to the second frame of reference using 3D reflected wave measurements received by the 3D reflected wave measuring device.

The processor may receive height data, the height data indicating a height of the device in the environment, and wherein the processor is configured to determine the height metric of the object in the environment using the height data and the 3D reflected wave measurements received by the 3D reflected wave measuring device.

In any of the above-mentioned embodiments, the 3D reflected wave measuring device may be a radar device. The orientation sensing device may comprise at least one of a gyroscope, a 3 axis accelerometer or a level sensor.

The device may comprise a fall detector.

The present invention also provides a method of using a device for measuring 3D coordinates associated with an object in an environment, the device comprising a 3D reflected wave measuring device for measuring 3D coordinates of an object in the environment, the 3D reflected wave measuring device having a 3D coordinate system having a first frame of reference, and an orientation sensing device to indicate an orientation of the 3D reflected wave measuring device in an environment for aligning the first frame of reference with a second frame of reference that is associated with the environment, the method comprising: installing the device in the environment by mounting the device on a wall; and aligning the first frame of reference with the second frame of reference.

In the above method, the device may be connected to one or more actuators for adjusting the orientation of the device in respective one or more axes, and wherein the step of aligning the first frame of reference with the second frame of reference comprises driving the one or more actuators in response to the orientation data to align the first frame of reference to the second frame of reference.

In an alternative method, the method may comprise outputting an indication of the orientation of the device in response to the orientation data, the indication for alerting a user at least one of audibly and visually. The indication may indicate: at least one of an angular direction and an angle in one or more axes in which the device should be moved in order to align the first frame of reference to the second frame of reference; and/or whether or not the device is aligned.

In the above methods, the step of aligning may be performed one or more times after installation of the device.

In a further alternative method, the step of aligning the first frame of reference with the second frame of reference comprises compensating for a difference between the first frame of reference and the second frame of reference using the orientation data.

In this further alternative method, compensating for a difference between the first frame of reference and the second frame of reference may comprise applying a rotation matrix to one or more axes of the first frame of reference and/or to one or more coordinates of 3D reflected wave measurements received by the 3D reflected wave measuring device.

In any of the above-mentioned methods, the method may further comprise determining a height metric of an object in the environment relative to the second frame of reference using 3D reflected wave measurements received by the 3D reflected wave measuring device.

The installed height of the device on the wall may be stored in memory in the device as height data, and the method comprises determining a height metric of the object in the environment relative to the second frame of reference using the height data and 3D reflected wave measurements received by the 3D reflected wave measuring device.

The step of aligning the first frame of reference with the second frame of reference may be performed at least one of one or more times after installation, or prior to each determination of the vertical displacement of an object in the environment

In any of the methods, the 3D reflected wave measuring device may be a radar device. The orientation sensing device may comprise at least one of a gyroscope, a 3 axis accelerometer or a level sensor.

In any of the methods, the device may comprise a fall detector.

Any instructions may be provided on one or more carriers. For example there may be one or more non-transient memories, e.g. a EEPROM (e.g. a flash memory) a disk, CD- or DVD-ROM, programmed memory such as read-only memory (e.g. for Firmware), one or more transient memories (e.g. RAM), and/or a data carrier(s) such as an optical or electrical signal carrier. The memory/memories may be integrated into a corresponding processing chip and/or separate to the chip. Code (and/or data) to implement embodiments of the present disclosure may comprise source, object or executable code in a conventional programming language (interpreted or compiled) such as C, or assembly code, code for setting up or controlling an ASIC (Application Specific Integrated Circuit) or FPGA (Field Programmable Gate Array), or code for a hardware description language.

These and other aspects will be apparent from the embodiments described in the following. The scope of the present disclosure is not intended to be limited by this summary nor to implementations that necessarily solve any or all of the disadvantages noted.

### BRIEF DESCRIPTION OF THE SEVERAL VIEWS OF THE DRAWINGS

The present invention will now be described, by way of example only, and with reference to the accompanying drawings, in which:

FIG. 1 illustrates an environment in which a device has been positioned;

FIG. 2 is a schematic block diagram of the device;

FIGS. 3a and 3b illustrates a human body with indications of reflections measured by a reflective wave detector when the person is in a standing non-fall state and in a fall state; and

FIGS. 4a and 4b illustrate the issue with misaligned frames of reference in a height metric determination.

### DETAILED DESCRIPTION

In the following detailed description, reference is made to the accompanying drawings that form a part hereof, and in which is shown by way of illustration specific embodiments in which the inventive subject matter may be practiced. These embodiments are described in sufficient detail to enable those skilled in the art to practice them, and it is to be understood that other embodiments may be utilized, and that structural, logical, and electrical changes may be made without departing from the scope of the inventive subject matter. Such embodiments of the inventive subject matter may be referred to, individually and/or collectively, herein by the term “invention” merely for convenience and without intending to voluntarily limit the scope of this application to any single invention or inventive concept if more than one is in fact disclosed.

The following description is, therefore, not to be taken in a limited sense, and the scope of the inventive subject matter is defined by the appended claims and their equivalents. In the following embodiments, like components are labelled with like reference numerals.

In the following embodiments, the term data store or memory is intended to encompass any computer readable storage medium and/or device (or collection of data storage mediums and/or devices). Examples of data stores include, but are not limited to, optical disks (e.g., CD-ROM, DVD-ROM, etc.), magnetic disks (e.g., hard disks, floppy disks, etc.), memory circuits (e.g., EEPROM, solid state drives, random-access memory (RAM), etc.), and/or the like.

As used herein, except wherein the context requires otherwise, the terms “comprises”, “includes”, “has” and grammatical variants of these terms, are not intended to be exhaustive. They are intended to allow for the possibility of further additives, components, integers or steps.

The functions or algorithms described herein are implemented in hardware, software or a combination of software and hardware in one or more embodiments. The software comprises computer executable instructions stored on computer readable carrier media such as memory or other type of storage devices. Further, described functions may correspond to modules, which may be software, hardware, firmware, or any combination thereof. Multiple functions are performed in one or more modules as desired, and the embodiments described are merely examples. The software is executed on a digital signal processor, ASIC, microprocessor, or other type of processor.

Specific embodiments will now be described with reference to the drawings.

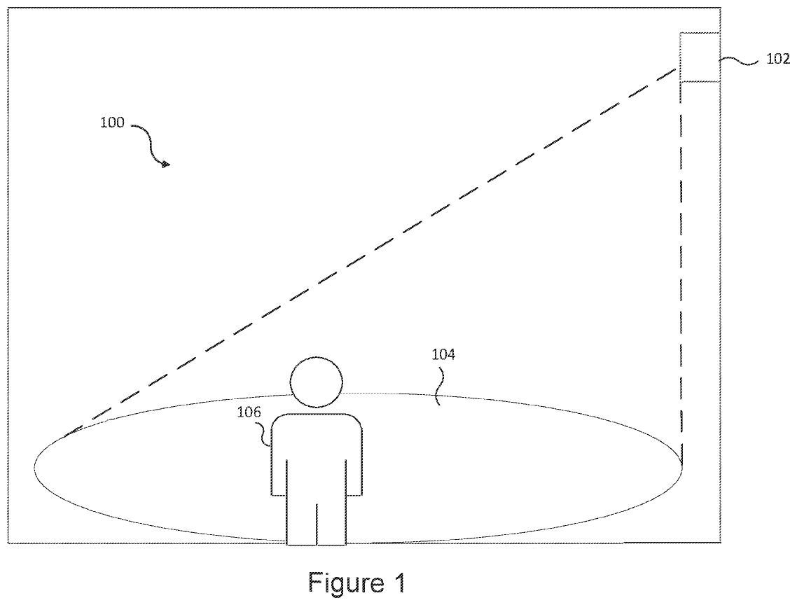

FIG. 1 illustrates an environment 100 in which a device 102 has been positioned. The environment 100 may for example be an indoor space such as a room of a home, a nursing home, a public building or other indoor space. Alternatively the environment may be an outdoor space such as a garden. The device 102 is configured to monitor a space 104 in the environment 100 in which a person 106 may be present.

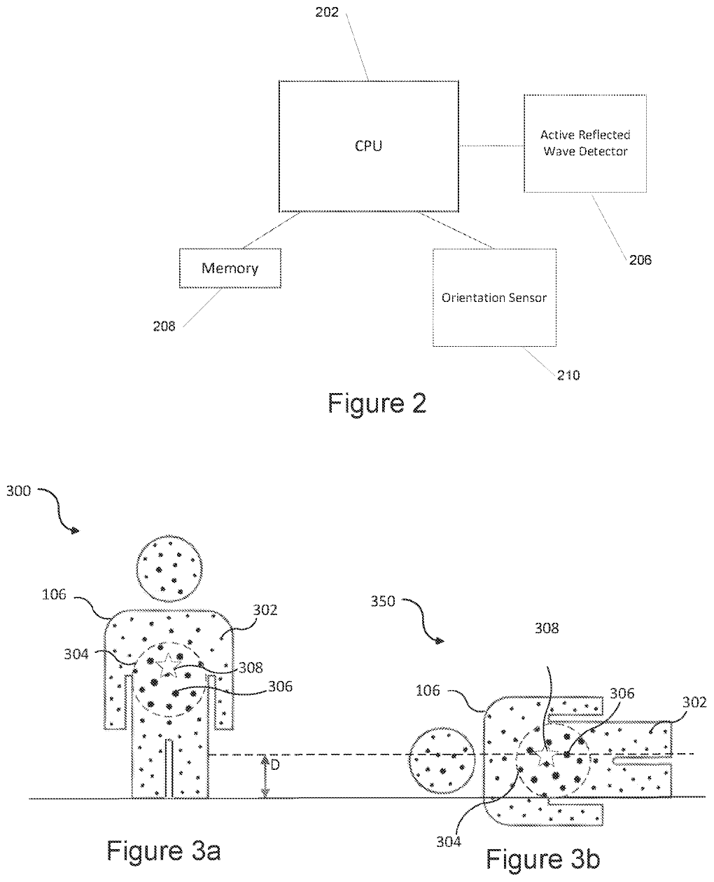

FIG. 2 illustrates a simplified view of the device 102. A shown in FIG. 2, the device 102 comprises a central processing unit (“CPU”) 202, to which is connected a memory 208. The functionality of the CPU 202 described herein may be implemented in code (software) stored on a memory (e.g. memory 208) comprising one or more storage media, and arranged for execution on a processor comprising on or more processing units. The storage media may be integrated into and/or separate from the CPU 202. The code is configured so as when fetched from the memory and executed on the processor to perform operations in line with embodiments discussed herein. Alternatively it is not excluded that some or all of the functionality of the CPU 202 is implemented in dedicated hardware circuitry, or configurable hardware circuitry like an FPGA.

FIG. 2 shows the CPU 202 being connected to an active reflected wave detector 206 (also referred herein as a 3D reflective wave measuring device) and an orientation sensing device 210. While in the illustrated embodiment the orientation sensing device 210 and reflected wave detector are separate from the CPU 202, in other embodiments, at least part of processing aspects of the active reflected wave detector 206 may be provided by a processor that also provides the CPU 202, and resources of the processor may be shared to provide the functions of the CPU 202 and the processing aspects active reflected wave detector 206. Similarly, functions of the CPU 202, such as those described herein, may be performed in the active reflected wave detector 206. The function and purpose of the orientation sensing device 210 will be described later.

As shown in FIG. 2, the device 102 may have a housing that houses the CPU 202, active reflected wave detector 206, orientation sensor 210 and memory 208. Alternatively, the active reflected wave detector 206 may be external to the device 102 and be coupled to the CPU 202 by way of a wired or wireless connection. Further, the outputs of the active reflected wave detector 206 may be wirelessly received from via an intermediary device that relays, manipulates and/or in part produces their outputs, for example a control hub of a monitoring and/or home automation system, which may in some cases comprise a security system.

In some embodiments, the CPU 202 is configured to detect the presence of a person in the monitored space 104, and if a person is present, classify the state or postural position (standing, sitting or having fallen) of the person based on an output of the active reflected wave detector 206.

The active reflected wave detector 206 may operate in accordance with one of various reflected wave technologies.

Preferably, the active reflected wave detector 206 is a radar sensor. The radar sensor 206 may use millimeter wave (mmWave) sensing technology. The radar is, in some embodiments, a continuous-wave radar, such as frequency modulated continuous wave (FMCW) technology. Such a chip with such technology may be, for example, Texas Instruments Inc. part number IWR6843. The radar may operate in microwave frequencies, e.g. in some embodiments a carrier wave in the range of 1-100 GHz (76-81 Ghz or 57-64 GHz in some embodiments), and/or radio waves in the 300 MHz to 300 GHz range, and/or millimeter waves in the 30 GHz to 300 GHz range. In some embodiments, the radar has a bandwidth of at least 1 GHz. The active reflected wave detector 206 may comprise antennas for both emitting waves and for receiving reflections of the emitted waves, and in some embodiment different antennas may be used for the emitting compared with the receiving.

The active reflected wave detector 206 is not limited to being a radar sensor, and in other embodiments, the active reflected wave detector 206 is a lidar sensor, or a sonar sensor.

The active reflected wave detector 206 being a radar sensor is advantageous over other reflected wave technologies in that radar signals can transmit through some materials, e.g. wood or plastic, but not others—notably water which is important because humans are mostly water. This means that the radar can potentially “see” a person in the environment 100 even if they are behind such an object. This is not the case for sonar.

In embodiments, the active reflected wave detector 206 is used to determine whether the person is in a posture that may be relate to them standing, sitting, or having fallen. This may be achieved for example by detecting a height associated with a certain location on their body, e.g. a location above their legs.

In operation, the active reflected wave detector 206 performs one or more reflected wave measurements at a given moment of time, and over time these reflected wave measurements can be correlated by the CPU 202 with the presence of a person and/or a state of the person and/or a condition of the person. In the context of the present disclosure, the state of the person may be a characterization of the person based on a momentary assessment. For example, a classification passed on their position (e.g. in a location in respect to the floor and in a configuration which are consistent or inconsistent with having fallen) and/or their kinematics (e.g. whether they have a velocity that is consistent or inconsistent with them having fallen, or having fallen possibly being immobile). In the context of the present disclosure, the condition of the person may comprise a determination of an aspect of the person's health or physical predicament, for example whether they are in a fall condition whereby they have fallen and are substantially immobile, such that they may not be able (physically and/or emotionally) to get to a phone to call for help. In some embodiments this involves an assessment of the person's status over time, such as in the order or 30-60 seconds. However, the condition of the person may in some contexts be synonymous with the status of the person. For example, by determining that the person is in a safe supported state or a standing state, it may be concluded that the person is not currently in a fall condition, whereby they are on the floor and potentially unable to seek help. It may additionally or alternatively be concluded that they are in a resting condition because of their status being determined to be in a safe supported state, e.g. lying on a bed. In another example their condition may be classified as active and/or mobile based on a determination of a walking status.

FIG. 3a illustrates a free-standing human body 106 with indications of reflective wave reflections therefrom in accordance with embodiments.

For each reflected wave measurement, for a specific time in a series of time-spaced reflective wave measurements, the reflective wave measurement may include a set of one or more measurement points that make up a “point cloud”. Each point 302 in the point cloud may be defined by a 3-dimensional spatial position from which a reflection was received, and defining a peak reflection value, and a doppler value from that spatial position. Thus, a measurement received from a reflective object may be defined by a single point, or a cluster of points from different positions on the object, depending on its size. In some embodiments the point cloud is prefiltered to exclude static points, for example by using a moving target indication as is known in the art.

FIG. 3a illustrates a map of reflections. The size of the point represents the intensity (magnitude) of energy level of the radar reflections (see larger point 306). Different parts or portions of the body reflect the emitted signal (e.g. radar) differently. For example, generally, reflections from areas of the torso 304 are stronger than reflections from the limbs. Each point represents coordinates within a bounding shape for each portion of the body. Each portion can be separately considered and have separate boundaries, e.g. the torso and the head may be designated as different portions. The point cloud can be used as the basis for a calculation of a reference parameter or set of parameters which can be stored instead of or in conjunction with the point cloud data for a reference object (human) for comparison with a parameter or set of parameters derived or calculated from a point cloud for radar detections from an object (human).

When a cluster of measurement points are received from an object in the environment 100, a location of a particular part/point on the object or a portion of the object, e.g. its centre, may be determined by the CPU 202 from the cluster of measurement point positions having regard to the intensity or magnitude of the reflections (e.g. a centre location comprising an average of the locations of the reflections weighted by their intensity or magnitude). As illustrated in FIG. 3a, the reference body has a point cloud from which its centre has been calculated and represented by the location 308, represented by the star shape. In this embodiment, the torso 304 of the body is separately identified from the body and the centre of that portion of the body is indicated. In alternative embodiments, the body can be treated as a whole or a centre can be determined for each of more than one body part e.g. the torso and the head, for separate comparisons with centres of corresponding portions of a scanned body.

The object's centre or portion's centre may be a weighted centre of the measurement points. The locations may be weighted according to an Radar Cross Section (RCS) estimate of each measurement point, where for each measurement point the RCS estimate may be calculated as a constant (which may be determined empirically for the reflected wave detector 206) multiplied by the signal to noise ratio for the measurement divided by R⁴, where R is the distance from the reflected wave detector 206 antenna configuration to the position corresponding to the measurement point. In other embodiments, the RCS may be calculated as a constant multiplied by the signal for the measurement divided by R⁴. This may be the case, for example, if the noise is constant or may be treated as though it were constant. Regardless, the received radar reflections in the exemplary embodiments described herein may be considered as an intensity value, such as an absolute value of the amplitude of a received radar signal.

In any case, the weighted centre, WC, of the measurement points for an object may be calculated for each dimension as:

$$WC = \frac{1}{{\sum }_{n = 1}^{N} {W}_{n}} {\sum }_{n = 1}^{N} ( {W}_{n} {P}_{n} )$$

### Where:

N is the number of measurement points for the object;
Wₙ is the RCS estimate for the n^{th} measurement point; and
Pₙ is the location (e.g. its coordinate) for the n^{th} measurement point in that dimension.

The CPU 202 may determine a height metric associated with at least one measurement of a reflection from the person conveyed in the output of the active reflected wave detector 206 and compare the height metric to at least one threshold.

The height metric may be a height of a weighted centre of the measurement points of a body or part thereof (where each measurement is weighted by the RCS estimation), and the CPU 202 may compare this height metric to a threshold distance, D, from the floor (e.g. 30 cm) and determine that the person in the environment is in a standing (non-fall) position if the height metric exceeds the threshold distance, this is illustrated in FIG. 3a.

The height metric is not limited to being a height of a weighted centre of the measurement points of the person's body or part thereof. In another example, the height metric may be a maximum height of all of the height measurements associated with the person's body or part thereof. In another example, the height metric may be an average height (e.g. median z value) of all of the height measurements of the person's body or part thereof. In the case of using a weighted centre or average height, the “part thereof” may beneficially be a part of the body that is above the person's legs to more confidently distinguish between fall and non-fall positions.

If the height metric (e.g. weighted centre, average height and/or maximum height) is within (less than) the threshold distance, D, from the floor (e.g. 30 cm), the CPU 202 may determine that the person in the environment is in a fall position, this is illustrated in FIG. 3b. If the height metric is greater than a first threshold distance from the floor but less than a second threshold distance from the floor (for example the a maximum height amongst the measurements associated with body is between 30 cm and 1.3 m, the CPU 202 may be able to detect that the person is in a safe reclined position, e.g. lying down on a bed or couch, which is an example of a non-fall position.

When the device is a fall detector, the detector could be a part of the device or a module of the code or part of its functionality.

### Alignment of the Active Reflective Wave Detector

As can be seen from the above methods, a reliable and accurate determination of the value for a height metric from the Active Reflective Wave Detector is important if a reliable determination of a position or posture of the person is to be made based on data received from the active reflected wave detector. That position may, for example, be a related to postural information indicative of a person standing position, sitting position or having fallen over. As mentioned above, such data may be in the form of sets of points defined by 3D coordinates and intensities or magnitudes of the reflective wave for each of the points.

The device may or may not be installed in an orientation and position such that its active reflected wave detector is not aligned with an expected frame of reference (for example a horizontal plane lying a height Hi above a point on a surface such as the floor, which in some embodiments is a horizontal surface). Thus, the expected frame of reference may have an axis x₂,y₂,z₂, which is associated with an environment. In particular, x₂ and y₂ are horizontal and z₂ is vertical, where the x₂-y₂ plane lies a distance H above the floor. A horizontal floor may thus lie in a plane defined by the equation z₂=−H. The device's active reflected wave detector has an actual frame of reference (i.e. how it is actually installed) having an axis x₁, y₁ and z₁, in which the z axis is only nominally (but not necessarily in practice) vertical. If the nominal axis (x₂, y₂, z₂) is not aligned with the expected frame of reference (x₁,y₁,z₁), both in terms of orientation (or at least the orientation of the z axis) and position (or at least the height position), then the device may incorrectly determine a height metric corresponding to coordinate associated with a measured reflect point. This is because the coordinate is measured with respect to the nominal frame of reference, but the height metric is with respect to the actual environment (i.e. a frame of reference of the environment; also referred to herein as an expected frame of reference).

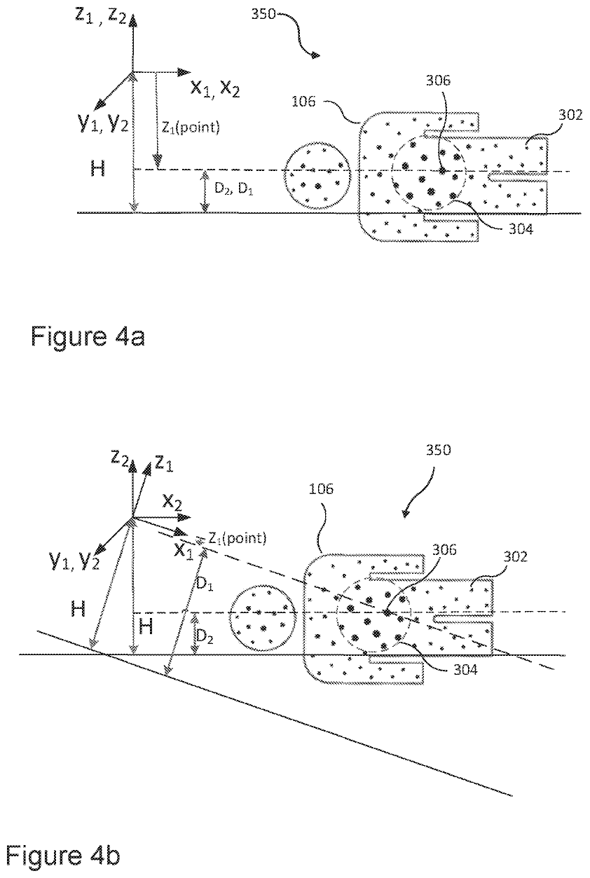

FIGS. 4a and 4b illustrate the issue with misaligned frames of reference in a height metric determination.

FIG. 4a, similar to FIG. 3b, shows a height metric determination as described above, where the active reflected wave detector is correctly aligned with the frame of reference to the height metric determination i.e. the floor. Using the measured reflected wave data from the active reflected wave detector, the vertical displacement of a point from a reference position such as the floor, and thus a height of that point from the floor, can be determined accurately. In this case shown in FIG. 4a, the reference body has a point cloud from that represents a particular measured point 306, amongst other points. The measured the point 306 is measured as being (with respect to the z axis) as being at z₁=−z₁(point). The height of the point 306 above the floor is calculated to be D₁, which equals H−z₁(point). In FIG. 4a, the device is mounted so that the active reflective wave detector's frame of reference of (x₁, y₁, z₁) is aligned with the environment's frame of reference (x₂, y₂, z₂). As a result, the calculated value of the height D₁ is equal to the actual height D₂.

FIG. 4b illustrates a scenario where the active reflected wave detector is incorrectly aligned within the environment. Misalignment could be angular misalignments in any one or more of x, y or z axes, and/or may include a positional misalignment of the respective origins of one or more axes. However, in the illustrated example, misalignment is in only in the z-axis. When mounted on a wall, the active reflected wave detector may be misaligned with respect to the z axis through rotation about the x axis (which also causes a misalignment with the y axis), and/or rotation about the y axis (which also causes misalignment with the x axis). FIG. 4b shows the latter case, where a rotation about the y axis has caused the x and z axes to be angularly misaligned such that the device is tilting forward away from the vertical wall defined by the z₂ axis.

Of course, misalignments may also occur in the x-y plane (where the device rotates about the z axis). However, misalignment by rotating in the z axis would not influence a vertical measurement (but simply misalign the useful field of view about the x-y axis). Therefore, such misalignments may be ignored in some embodiments.

In the case of the in FIG. 4b, the calculated height D₁ of the measured point 306 with respect to the active reflected wave detector's frame of reference is not equal to the actual height D₂, so is incorrect. If a determination of a condition or position of a person is taken based on a comparison of D₁, treated as the height metric, with a threshold value, this could cause a system implementing this device as a detector such as a fall detector to incorrectly determine that the person 302 is not in the fall position indicated in FIG. 4b, or in another fall position. In other scenarios a fall detector may determine that the person is in a fall position when they are not.

As will be appreciated, in addition to or instead of using the height D₁ of the point 306 as the height metric, the height metric used for the comparison with the threshold may be associated based on one or more points associated with the person 106. For example, the height metric could correspond to a spatial position of a weighted centre of the measurements (308 in FIG. 2) reflected from the person 106.

The embodiments disclosed herein facilitate or cause alignment of the 3D reflected wave measuring device (i.e. the active reflected wave detector 206) with a frame of reference of the environment. This is done using data from the orientation sensing device 210, which, in practice may be at least one of a gyroscope, a 3 axis accelerometer and a level sensor. In some embodiments the orientation sensing device 210 be 6-axis orientation sensing devices, the 6 axis consisting of 3 from a gyroscope and 3 from an accelerometer.

There are two main implementations of an alignment correction of the device in order that the device is correctly aligned to a frame of reference within the environment: 1) physical alignment; and 2) mathematical alignment. In either case, the purpose of the alignment correction is to re-align the orientation of the z-axis of the device with the z axis of the frame of reference in the environment, where misalignment has occurred because of a rotation of the device about the x and/or y axes. The alignment correction may also, or instead, be performed to re-align the x and/or y axes in the case where there has been a rotation of the device about the z axis. However, in some embodiments, misalignment in the x and/or y axes as a result of rotation of the device about the z axis is ignored.

The physical alignment is discussed first.

In this embodiment, and in its most fundamental configuration, the device utilises the orientation sensor 210 for the processor to provide feedback for a user to manually adjust a position of the device when installing the device.

Most simply the indication could be provided by one or more level meters (or representations of level meters). Two level meters respectively lying in perpendicular planes are preferable. As such, the user would adjust the pan and tilt of the device during installation in order to achieve the desired position as indicated by the level meters. The indication could in its most basic form be a binary output of level (to within a predefined accuracy) or not level.

In a related embodiment, the processor may give an indication when out of alignment of an extent and preferably also direction of the misalignment so that a person could easily align the device as needed. The indication may be provided by a visual and/or audio means on the device itself, or communicated to another device, for example a smartphone, tablet or computer.

In alternative embodiment where physical re-alignment occurs, the processor 202 may be coupled to drive actuators that control the position (pan and tilt) of the device. A closed loop control system may be implemented where the processor utilises the data from the orientation sensing device 210 in order to drive the actuators to move the device to minimise the misalignment between the device and the frame of reference (for example a horizontal plane lying at height Hi above a point on a surface such as the floor, which in some embodiments is a horizontal surface).

The alignment of the device may be performed one or more times after installation of the device. The device may alert the user periodically if the position of the device has become misaligned with the intended frame of reference. With the motorised embodiment, the device may continuously monitor its orientation relative to the intended frame of reference.

Now discussed is the mathematical method of aligning the frame of reference of the orientation sensing device with the frame of reference of the environment.

In this embodiment, which is the preferred embodiment, the device is not physically reoriented, but rather, the angular difference (at least with respect to the z axis) between the second frame of reference and the first frame of reference (of the orientation sensing device) is compensated for so that the measurements made with respect to the first frame of reference are interpreted with respect to the second frame of reference.

The compensation can be achieved, for example using a rotation matrix. For example, the rotation matrix can be applied to either coordinates of respective measured wave reflections from the 3D reflected wave device, or to the first frame of reference.

Rotation matrices are well known in the in the operation of 3D coordinate systems.

Once the rotation matrix has been applied, the measurements made by the 3D reflected wave device are aligned to the intended frame of reference. Height metric measurements made based on the reflected wave measurements therefore provides an accurate determination of the height metric of an object, irrespective of the physical orientation of the device once installed.

The compensation may occur periodically, or before each determination of a height metric. It is also noted that the compensation, may be applied firstly on the raw data being received from the active reflective wave detector before any processing of the data to determine a weighted centre point is performed. Alternatively, the raw data may be processed to determine a weighted centre point (or other spatial location based on the raw data), and then compensation may be applied.

In either of the above implementations of the alignment, installation height data (H) may be stored in the memory of the device. The installation height data may comprise a height value at which the device is instructed to be installed on a wall. Alternatively installation height data may be data that is input by the installer of the device once the device has been installed. Furthermore, the installation height data may be data that is measured using the 3D reflected wave detector 206 (once correctly aligned with the intended frame of reference), for example based on a measured z-displacement from one or more identifiable reflection points on the floor, for example by a retroreflector placed on, or moving along, the floor.

As such, the vertical displacement of the 3D measurements with respect to the second frame of reference may be determined based on a vertical difference between the reflected data from the object and the installation height data.

Where it is referred herein to “aligning the frame of reference of the 3D reflected wave measuring device”, this means aligning the frame of reference of the 3D reflected wave measuring device 206. It is the orientation sensing device 210 that provides the orientation data. However, the orientation sensing device package has a fixed and known positional relationship with respect to the 3D reflected wave measuring device, and so the orientation of the 3D reflected wave measuring device 206 may be determined with respect to the orientation sensing device 210 to that any differences between their respective orientations may be calibrated out (e.g. by mathematical alignment such as by using a rotation matrix, for example as described herein). Such a calibration may be performed in during manufacture of the device 102 for example, prior to its installation for use in the environment. It will be appreciated, however, that the aligning the frame of reference of the 3D reflected wave measuring device 206 to the frame of reference associated with the environment may be equivalently performed by aligning the orientation of the sensor sensing device 210 with the frame of reference of the environment, either physically or by mathematical manipulation, in conjunction with aligning the orientation of the sensor sensing device 210 with the frame of reference of the environment 3D reflected wave measuring device 206, either physically or by mathematical manipulation.

The phrase “A and/or B”, “at least one of A and B” and “at least one of A or B” are each intended to include the following options, unless the contexts clearly requires otherwise: “A”, “B”, “at least A”, “at least B”, “at least one of A”, “at least one of B”, and all combinations thereof relating A with B, for example, “at least one of A and at least one of B”. Where used in a claim, the feature of the claim should be taken to be fulfilled if any one or more of those options is satisfied. If in a particular context any of those options are nonsensical or result in a duplicated option then, in that particular context, only the nonsensical option or duplicated option may be disregarded.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A fall detector device for determining a height metric of an object in an environment using 3D coordinates associated with the object in the environment for determining a fall condition of the object, comprising:
a 3D reflected wave measuring device having a 3D coordinate system for measuring 3D coordinates of an object in the environment for determining the height metric of the object in the environment, the 3D reflected wave measuring device having a first frame of reference;
an orientation sensing device to indicate an orientation of the 3D reflected wave measuring device in an environment for aligning the first frame of reference with a second frame of reference that is associated with the environment;
a processor coupled to the orientation sensing device and configured to receive orientation data indicative of an orientation of the device, the processor being configured to align the first frame of reference with the second frame of reference by using the orientation data to compensate for an angular difference between the first frame of reference and the second frame of reference,
wherein the fall detector device is mountable on a wall within the environment and the second frame of reference comprises a horizontal plane associated with the environment, and
wherein the processor is configured to determine the height metric of the object in the environment relative to the second frame of reference using 3D reflected wave measurements received by the 3D reflected wave measuring device.

2.-6. (canceled)

7. A fall detector device according to claim 1, wherein the processor is configured to compensate for a difference between the first frame of reference and the second frame of reference by applying a rotation matrix to one or more axes of the first frame of reference and/or to one or more coordinates of 3D reflected wave measurements received by the 3D reflected wave measuring device.

8. (canceled)

9. A fall detector device according to claim 1, wherein the processor receives height data, the height data indicating a height of the device in the environment, and wherein the processor is configured to determine the height metric of the object in the environment using the height data and the 3D reflected wave measurements received by the 3D reflected wave measuring device.

10. A fall detector device according claim 1, wherein the 3D reflected wave measuring device is a radar device.

11. A fall detector device according to claim 1, wherein the orientation sensing device comprises at least one of a gyroscope, a 3 axis accelerometer or a level sensor.

12. A method of using a fall detector device for determining a height metric of an object in an environment using 3D coordinates associated with the object in the environment for determining a fall condition of the object,
the device comprising:
a 3D reflected wave measuring device for measuring 3D coordinates of an object in the environment for determining the height metric of the object in the environment, the 3D reflected wave measuring device having a 3D coordinate system having a first frame of reference;
an orientation sensing device to indicate an orientation of the 3D reflected wave measuring device in an environment for aligning the first frame of reference with a second frame of reference that is associated with the environment;
a processor coupled to the orientation sensing device and configured to receive orientation data indicative of an orientation of the device,
the method comprising:
installing the device in the environment by mounting the device on the wall, wherein the second frame of reference comprises a horizontal plane associated with the environment;
aligning the first frame of reference with the second frame of reference using the orientation data to compensate for an angular difference between the first frame of reference and the second frame of reference; and
determining the height metric of the object in the environment relative to the second frame of reference using 3D reflected wave measurements received by the 3D reflected wave measuring device.

13.-15. (canceled)

16. A method according to claim 12, wherein the step of aligning is performed one or more times after installation of the fall detector device.

17. (canceled)

18. A method according to claim 12, wherein compensating for a difference between the first frame of reference and the second frame of reference comprises applying a rotation matrix to one or more axes of the first frame of reference and/or to one or more coordinates of 3D reflected wave measurements received by the 3D reflected wave measuring device.

19. (canceled)

20. A method according to claim 12, wherein the installed height of the fall detector device on the wall is stored in memory in the device as height data, and the method comprises determining a height metric of the object in the environment relative to the second frame of reference using the height data and 3D reflected wave measurements received by the 3D reflected wave measuring device.

21. A method according to claim 12, wherein the step of aligning the first frame of reference with the second frame of reference is performed at least one of one or more times after installation, or prior to each determination of the vertical displacement of an object in the environment.

22. A method according to claim 12, wherein the 3D reflected wave measuring device is a radar device.

23. A method according to claim 12, wherein the orientation sensing device comprises at least one of a gyroscope, a 3 axis accelerometer or a level sensor.

24. A fall detector device according to claim 1, wherein the height metric determined by the processor comprises a height of a weighted centre of the 3D reflected wave measurements received by the 3D reflected wave measuring device.

25. A fall detector device according to claim 24, wherein the height of the weighted centre of the 3D reflected wave measurements received by the 3D reflected wave measuring device is based on a Radar Cross Section (RCS) of each 3D reflected wave measurement.

26. A fall detector device according to claim 1, wherein the height metric determined by the processor comprises a maximum height of the 3D reflected wave measurements received by the 3D reflected wave measuring device.

27. A fall detector device according to claim 1, wherein the height metric determined by the processor comprises an average height of the 3D reflected wave measurements received by the 3D reflected wave measuring device.

28. A method according to claim 20, wherein the height metric comprises a height of a weighted centre of the 3D reflected wave measurements received by the 3D reflected wave measuring device.

29. A method according to claim 28, wherein the height of the weighted centre of the 3D reflected wave measurements received by the 3D reflected wave measuring device is based on a Radar Cross Section (RCS) of each 3D reflected wave measurement.

30. A method according to claim 20, wherein the height metric comprises a maximum height of the 3D reflected wave measurements received by the 3D reflected wave measuring device.

31. A method according to claim 20, wherein the height metric comprises an average height of the 3D reflected wave measurements received by the 3D reflected wave measuring device.
